(19) United States
(12) Patent Application Publication
(10) Pub. No.: US 2021/0251802 A1
(43) Pub. Date: Aug. 19, 2021

(54) UTILITY GARMENT WITH THERAPEUTIC CHARACTERISTICS

(71) Applicant: Light Tree Ventures Holding B.V., RIJSWIJK (NL)

(72) Inventor: ALAIN DIJKSTRA, Amstelveen (NL)

(73) Assignee: Light Tree Ventures Holding B.V., RIJSWIJK (NL)

(21) Appl. No.: 16/792,895

(22) Filed: Feb. 18, 2020

(51) Int. Cl. A61F 7/02; A41D 1/00; A61N 5/06

(52) U.S. Cl. CPC A61F 7/02; A41D 1/002; A61N 5/0625; A61N 5/0624; A61N 2005/0659; A61F 2007/0086; A61F 2007/0238; A61F 2007/0292; A61N 2005/0652; A61N 2005/0653; A61F 2007/026; A61N 2005/0645

(57) ABSTRACT

A utility garment comprises a first layer and a second layer of a fabric, an infrared heater including a plurality of flexible heating elements, provided between the first layer and the second layer of the fabric, a plurality of Light Emitting Diodes (LEDs) provided between the first and the second layer of the fabric and a plurality of sensors configured to determine a plurality of parameters pertaining to a body by which the utility garment has been worn

### TECHNICAL FIELD

The present invention relates generally to utility garments such as those used during physical activities such as physical training, sports, and yoga, etc. More specifically the present invention relates to utility garments with therapeutic characteristics facilitated through surface heating means and radiation emitters.

### BACKGROUND ART

Utility garments such as sportswear and physical training suits have been known in the art for some time. Increasingly, such garments have been provided with some additional features such as motion and location sensors for tracking and performance measurements. However, such garments are generally used in laboratory environments for detailed performance studies pertaining typically to professional athletes.

Even though a few products, in that regard, are also commercially available for amateur everyday usage, they are generally available with rudimentary features such as GPS monitoring through a mobile application. Logic is made available in the mobile application generally for additional measurements such as distance covered and calories burned determined from internally coded correlation functions. More recently some products such as therapeutic bandages have been introduced with radiation emitting sources such as Light Emitting Diodes (LEDs) that are being used for treatment of conditions such as soft tissue injuries, edema, wound healing, nerve damage, acne, and joint pain, etc. Such therapeutic bandages are generally very treatment specific and are not designed for regular everyday usage. Some of the solutions, in that regard, suggested in state of the art include the following documents.

WO2016009277A1 discloses a method of making garments (including compression garments) having one or more highly stretchable conductive ink pattern formed of a composite of an insulated adhesive, a conductive ink, and an intermediate gradient zone between the adhesive and conductive ink where stretchable conductive ink patterns may be stretched more than twice their length without breaking or rupturing. Also, the flexible garment is provided with flexible battery and/or modular battery for transferring heat to conductive ink and provided therapeutic effect on user body. Also a body sensor may detect user movement (e.g., movement of individual body parts (arms, legs, etc.) and/or movement of the entire user (e.g., rate of motion, direction of motion, altitude, etc.). However, conductive ink patterns are limited in their capacity for heat transfer. Moreover, the garment has not been provided with any provision for providing Photo-Dynamic Therapy (PDT).

U.S. Pat. No. 9,869,807B2 discloses a wearable device including clothing, a strap for a bag or a watch. The wearable device contains an array of one or more different types of electrical components. Components include light-emitting diodes (e.g., micro-light-emitting diodes), organic light-emitting diodes, vibrators or other electrically controlled actuators, sensors, and/or other electrical devices. To accommodate deformation (e.g., bending and/or stretching in one or more dimensions), the wearable device formed by mounting an array of components (light-emitting diodes) to a flexible substrate. The flexible substrate has signal paths that accommodate the deformation of the substrate without cracking. The flexible substrate and components (light-emitting diodes) have been mounted on the support structure. The structure may be a layer of plastic, metal, glass, sapphire or other crystalline material, ceramic, fabric, or other material. Adhesive tape may be used in attaching flexible substrate to Structure layer or flexible substrate may be attached to the structure layer using heat and/or pressure. However, the document does not disclose provisions for heating of body of a user for applications such as enhanced blood flow and pain relief.

In either of the aforementioned documents and other documents present in the state of the art, specific structures and features that may be customizable for different body structures have not been disclosed. Moreover, neither of the documents attempts to provide flexibility in the utilization of the wearable inventions in order to adapt them for varying applications and environments.

Therefore there is a need in the art for a utility garment that does not suffer from the aforementioned deficiencies.

### SUMMARY OF THE INVENTION

According to a first aspect of the present invention, there is provided a utility garment, comprising a first layer and a second layer of a fabric, an infrared heater including a plurality of flexible heating elements, provided between the first layer and the second layer of the fabric, a plurality of Light Emitting Diodes (LEDs) provided between the first and the second layer of the fabric and a plurality of sensors configured to determine a plurality of parameters pertaining to a body by which the utility garment has been worn.

In one embodiment of the invention, the plurality of sensors and the plurality of LEDs are connected through a plurality of conductors arranged in one or more predetermined patterns.

In one embodiment of the invention, the plurality of conductors are woven in the fabric.

In one embodiment of the invention, the plurality of conductors are flexible threads of an electrically conducting material and connecting terminals of the plurality of LEDs are pliable so as to allow the connecting terminals to form a loop around the passing plurality of conductors.

In one embodiment of the invention, the plurality of LEDs are provided on one or more of flexible Organic LED (OLED) and inorganic LED based films.

In one embodiment of the invention, the plurality of flexible heating elements are located in a plurality of respective grooves in the fabric and a predetermined amount of clearance has been provided in the plurality of grooves of the fabric, to allow for the adjustment of the plurality of flexible heating elements.

In one embodiment of the invention, the plurality of LEDs are connected in one or more of series connections, parallel connections, and combinations thereof.

In one embodiment of the invention, the utility garment further comprises a plurality of auxiliary fabric portions for covering joint areas in the body.

In one embodiment of the invention, the plurality of auxiliary fabric portions includes a plurality of auxiliary infrared heating elements.

In one embodiment of the invention, the plurality of auxiliary fabric portions are stretchable in nature and are provided in one or more of straight, zigzag and coiled forms or combinations thereof.

In one embodiment of the invention, each one of the plurality of auxiliary fabric portions includes a respective vacant portion, in order to prevent included plurality of LEDs from relocating over to joint lines.

In one embodiment of the invention, the plurality of flexible heating elements are arranged in order to align with a plurality of veins in the body.

In one embodiment of the invention, the utility garment further comprises a plurality of additional pockets in order to receive one or more of additional heating and cooling packages.

In one embodiment of the invention, the one or more of additional heating and cooling packages include Phase Change Materials (PCMs).

In one embodiment of the invention, the plurality of additional pockets includes power connectors in order to supply power to the received one or more of additional heating and cooling packages.

In one embodiment of the invention, the utility garment further comprises a plurality of moisture absorbing portions, each one of the plurality of moisture absorbing portions including a padding material adapted to arrest sweat through one or more of absorption and adsorption and a plurality of anti-microbial LEDs adapted to irradiate the body for germicidal applications.

In one embodiment of the invention, the infrared heater, the plurality of LEDs and the plurality of sensors are connected with a processor and a memory unit, the memory unit including machine readable instructions that when executed by the processor, enables the processor to determine magnitudes of the plurality of parameters through connection with the plurality of sensors and regulate the infrared heater and the plurality of LEDs in correlation with the determined magnitudes.

In one embodiment of the invention, the processor is further enabled to regulate a plurality of treatment parameters through the regulation of the infrared heater and the plurality of LEDs.

In one embodiment of the invention, the processor is further enabled to communicate with a handheld device through one or more a long range communication network and a short range communication network.

In one embodiment of the invention, the processor is further enabled to receive a control signal from the handheld device in order to set the plurality of treatment parameters within low, medium and high ranges.

In one embodiment of the invention, the utility garment further comprises a power source configured to provide electrical power to the infrared heater, the plurality of LEDs and the plurality of sensors.

In one embodiment of the invention, the power source includes a Thermo-Electric Generator (TEG) based power source.

In one embodiment of the invention, TEG based power source includes Eutectic Gallium Indium (EGaIn) liquid metal interconnects encased in High Thermal Conductivity (HTC) elastomers.

In one embodiment of the invention, the HTC elastomers are doped with graphene.

According to a second aspect of the present invention, there is provided a method of utilizing a utility garment, the utility garment including an infrared heater, a plurality of LEDs and a plurality of sensors, the method comprising steps of determining magnitudes of a plurality of parameters pertaining to a body by which the utility garment has been worn, through connection with the plurality of sensors and regulating the infrared heater and the plurality of LEDs in correlation with the determined magnitudes.

### BRIEF DESCRIPTION OF ACCOMPANYING DRAWINGS

So that the manner in which the above recited features of the present invention can be understood in detail, a more particular description of the invention, briefly summarized above, may have been referred by embodiments, some of which are illustrated in the appended drawings. It is to be noted, however, that the appended drawings illustrate only typical embodiments of this invention and are therefore not to be considered limiting of its scope, for the invention may admit to other equally effective embodiments.

These and other features, benefits, and advantages of the present invention will become apparent by reference to the following text figure, with like reference numbers referring to like structures across the views, wherein:

FIG. 1A illustrates an exploded partial view of a utility garment, in accordance with an embodiment of the present invention;

FIG. 1B illustrates an exemplary arrangement of a plurality of flexible heating elements of an infrared heater, in accordance with an embodiment of the present invention;

FIG. 1C illustrates connection assembly of a Light Emitting Diode (LED) with a plurality of conductors, in accordance with an embodiment of the present invention;

FIG. 1D illustrates connection arrangements of a plurality of LEDs, in accordance with an embodiment of the present invention;

FIG. 2 illustrates two exemplary arrangements of the plurality of conductors, within the utility garment, in accordance with an embodiment of the present invention;

FIG. 3A illustrates the utility garment in accordance with another embodiment of the present invention;

FIG. 3B illustrates the utility garment in accordance with yet another embodiment of the present invention;

FIG. 3C illustrates the utility garment of FIG. 3B, in accordance with yet another embodiment of the present invention;

FIG. 3D illustrates the utility garment of FIG. 1B, in accordance with yet another embodiment of the present invention;

FIG. 4 illustrates a logical diagram of control architecture provided in the utility garment, in accordance with an embodiment of the present invention;

FIG. 5 illustrates a Thermo-Electric Generator (TEG) based power source for the utility garment, in accordance with an embodiment of the present invention;

FIG. 6 illustrates a system for a handheld device based control of the utility garment, in accordance with an embodiment of the present invention; and

FIG. 7 illustrates a system for a handheld device based control of the utility garment, in accordance with another embodiment of the present invention.

### DETAILED DESCRIPTION

While the present invention is described herein by way of example using embodiments and illustrative drawings, those skilled in the art will recognize that the invention is not limited to the embodiments of drawing or drawings described, and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claims. As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes. Any discussion of documents, acts, materials, devices, articles and the like is included in the specification solely for the purpose of providing a context for the present invention. It is not suggested or represented that any or all of these matters form part of the prior art base or were common general knowledge in the field relevant to the present invention.

In this disclosure, whenever a composition or an element or a group of elements is preceded with the transitional phrase “comprising”, it is understood that we also contemplate the same composition, element or group of elements with transitional phrases “consisting of”, “consisting”, “selected from the group of consisting of”, “including”, or “is” preceding the recitation of the composition, element or group of elements and vice versa.

The present invention is described hereinafter by various embodiments with reference to the accompanying drawings, wherein reference numerals used in the accompanying drawing correspond to the like elements throughout the description. This invention may, however, be embodied in many different forms and should not be construed as limited to the embodiment set forth herein. Rather, the embodiment is provided so that this disclosure will be thorough and complete and will fully convey the scope of the invention to those skilled in the art. In the following detailed description, numeric values and ranges are provided for various aspects of the implementations described. These values and ranges are to be treated as examples only, and are not intended to limit the scope of the claims. In addition, a number of materials are identified as suitable for various facets of the implementations. These materials are to be treated as exemplary, and are not intended to limit the scope of the invention.

It is envisaged here that a utility garment may be provided for several possible applications such as sports, physical training, outdoor relaxation, and yoga etc. For example, the utility garment may be a track-suit or a track pant or a head gear or a shirt or a jacket etc. The utility garment may be able to sense body parameters such as temperature, perspiration, heart rate and velocity of a user and provide heating and electromagnetic radiation based therapy through a built-in infrared heater and a plurality of therapeutic Light Emitting Diodes (LEDs), respectively. In that regard, it is further envisaged that the infrared heater may be provided in form of resistive carbon or ceramics or a blend of carbon and ceramic based flexible fibers that may be powered by an onboard power source. The heat transfer is envisaged to be through infrared electromagnetic radiation having wavelengths typically ranging from 700 nanometers to 1 millimeter. The on-board power source may be a rechargeable or replaceable battery, such as Lithium based batteries, or may be based on upcoming power generating technologies involving Thermo-Electric Generators (TEG) that utilize body heat to generate the power.

Fabric materials used for the utility garment are envisaged to be stretchable and flexible for ease of utilization. The fabric materials are also envisaged to be water resistant, capable of shielding residual electromagnetic field for the proper functioning of electronic components and capable of dissipating static electricity for preventing the static electricity to be passed onto the skin of the user. Moreover, conductors connecting the electronic components such as sensors, LEDs, processors, and memory units are envisaged to include sufficient slack in form of ‘S’ shape or zigzag pattern in order to accommodate the stretching and release of the fabric. Moreover, LEDs are envisaged to be directly connected to a conductive material (such as silver) based threads, wherein terminals of the LEDs are forming loops around the conductive threads in order to eliminate the need for use of relatively rigid Printed Circuit Boards (PCBs) for mounting the LEDs. Moreover, the conductive threads may be sewn with the fabric of the utility garment. Alternately, LEDs may be provided on flexible Organic LED (OLED) films eliminating the need for rigid LEDs rendering even more flexibility to the utility garment. As another alternative, flexible inorganic LED light sheets may also be utilized for the same purposes, as will be illustrated in the following discussion.

Several other constructional features may be provided in the utility garment in order to increase the overall usability value of the utility garment. For example, grooves may be provided in the utility garment in order to align the flexible heating fibers with veins of the user for enhanced blood flow. Moreover, clearances may be provided in the grooves accommodating the flexible heating fibers so that the fibers can be relocated based on specific body anatomy of a different user. Additional heating may be provided in joint areas through additional flexible patches made of insulating material and including comparatively higher heating fiber density. In addition, the utility garment may be provided with additional pockets at several locations, in order to accommodate additional heating or cooling packages. Such packages may include Phase Change Materials (PCMs) such as water or urea for passive heating or cooling. The packages may also be resistive, optical or Peltier effect based on active heating or cooling. In that regard, power connectors may be provided in the additional pockets to power active packages. Further, in areas of the utility garment, which cover portions of the body that are susceptible to excessive sweating, additional moisture absorbing portions may be provided. Such moisture absorbing portions may additionally include padding material in order to arrest the sweat and anti-microbial LEDs for their germicidal properties.

In any case, the utility garment is envisaged to be a smart garment and has therefore been provided with control architecture including one or more processors and memory units. Logic built into the control architecture would allow the processors to control several treatment parameters such as intensity and dosage of heat and LED radiation, wavelengths of the LED radiation, mode of operation (pulsing or continuous), time duration and combinations thereof. However, in addition to automatic control of treatment parameters, the user would be able to regulate them through a handheld device, such as a smartphone, by connecting with the utility garment over a Wide Area Network (WAN) such as Internet or through short range communication such as Bluetooth, Near Field Communication (NFC) or ZigBee etc. Referring to the figures now, the invention will be explained in further details.

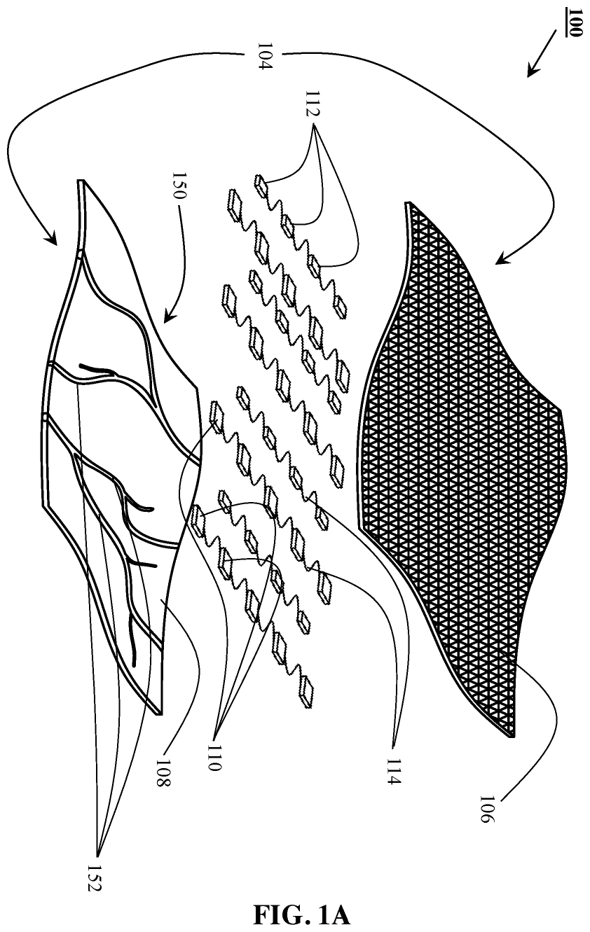

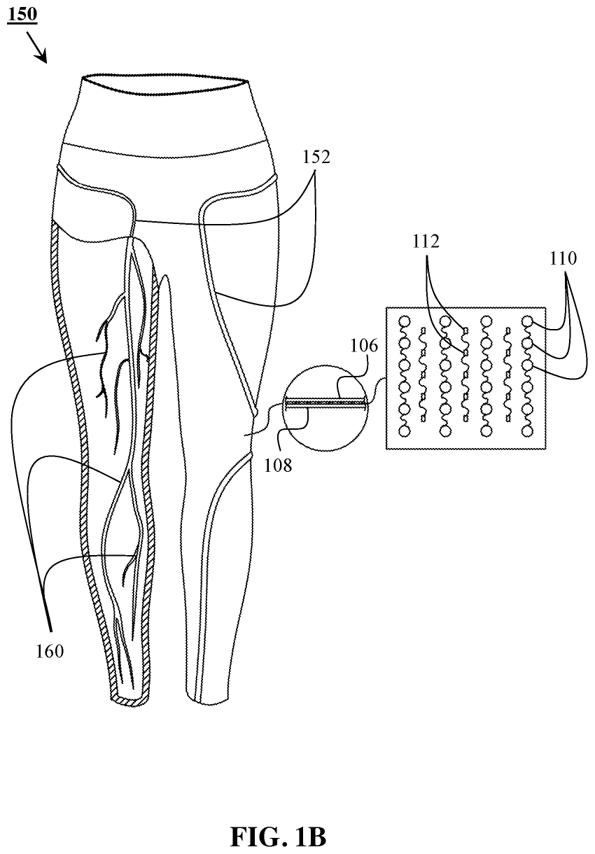

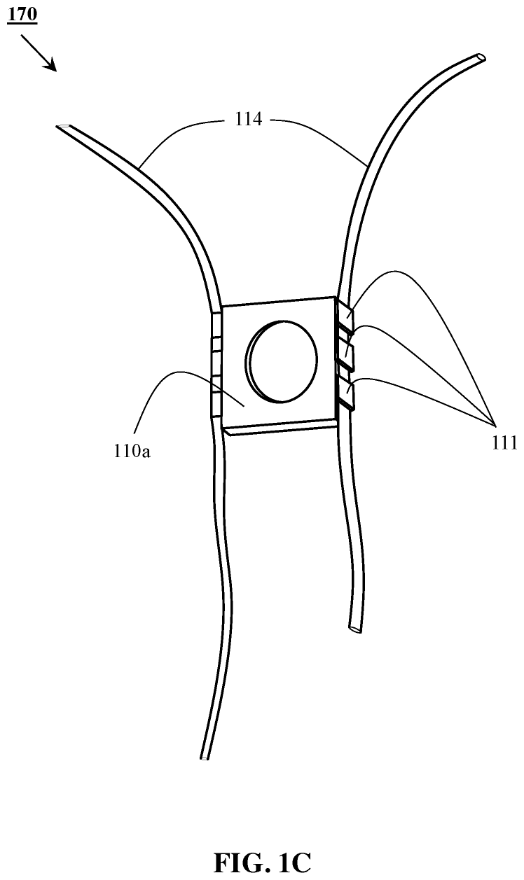

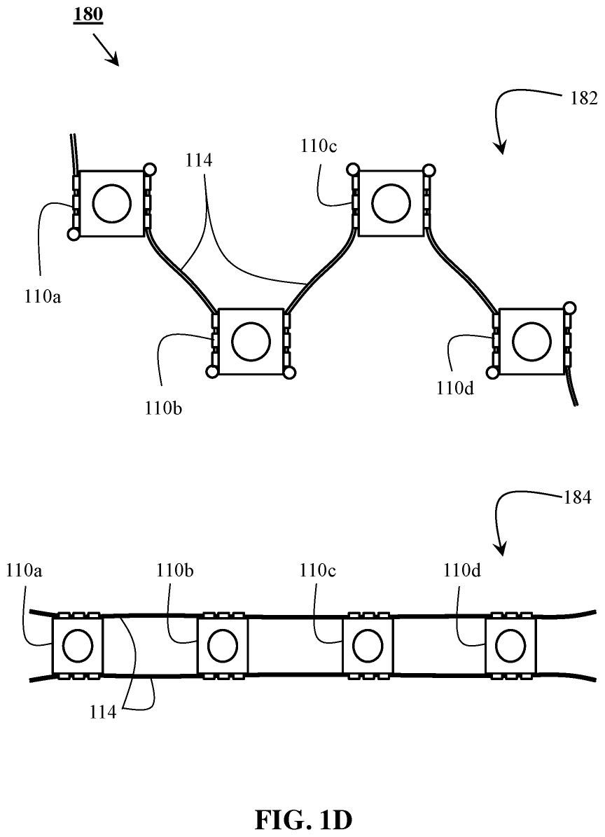

FIG. 1A illustrates a partial exploded view of a utility garment 100, in accordance with an embodiment of the present invention. The utility garment 100 may be a track-suit, a track pant, a head gear, a shirt or a jacket etc. As shown in FIG. 1A, the utility garment 100 includes a first layer 106 and a second layer 108 of a fabric 104. In that regard, the fabric 104 may be any woven, non-woven, knit or otherwise created flexible sheet material that is appropriate for manufacturing garments. Alternately, the first layer 106 and the second layer 108 may also be made from distinct fabric types. For example, the first layer 106 that is exposed to environment may be made from a relatively resistant fabric, having insulating characteristics, such as polyester, polyamide, polyaramid, polytetrafluorethylene, polyethylene, polypropylene, polyurethane, silicone, mixtures of polyurethane and polyethyleneglycol, ultrahigh molecular weight polyethylene, high-performance polyethylene, nylon, LYCRA and the like. Alternately, hydrophilic surfactants may be provided on surfaces of the fibers to generate a wicking effect. However, the fabric for the second layer 108 may be compatible with several skin types and may vary from user to user, based on the sensitivity of the skin of a user in generating an allergic response. In that regard, the second layer 108 may be made from cotton, wool, silk, nylon, polyester or other synthetic materials. It is envisaged here that the fabric 104 may be a stretchable fabric in order to facilitate ease of use and movement for a user.

Moreover, outer surfaces of the first layer 106 and the second layer 108 that are directed away from the skin of the user, may also be provided with water repelling agents in interstitial spaces between fibers of the fabric 104, in order to prevent moisture in any form from reaching the electronic components of the utility garment 100. In several other embodiments, the first layer 106 and the second layer 108 may also be provided with coatings including aqueous solutions of pure silk fibroin based protein segments. Silk fibroin based protein segments provide improved moisture management properties, resistance to microbial growth, increased abrasion and thermal resistance, etc. More information on the same can be found in a publication of United States Patent Application Numbered US20190003113A1, titled “Silk Performance Apparel and Products and Methods of Preparing the Same”, that is included herein by reference, in its entirety. Further, inner surfaces of the second layer 108 may be polymerized with conductive compounds on fiber surfaces for dissipation of static electricity generated by friction or the like.

The utility garment 100 also includes a plurality of Light Emitting Diodes (LEDs) 110 provided between the first layer 106 and the second layer 108. The plurality of LEDs 110 may in that manner utilize several wavelengths of light, such as, but not limited to, infrared (700 nm to 1 mm), visible light (380 to 700 nm) and Ultra-Violet (UV) (10 to 400 nm), such as for acne treatment with blue light (400 to 470 nm) and as an anti-aging agent with red (630 to 700 nm). Different wavelengths can be obtained from LEDs made from pure or doped semiconductor materials. Commonly used semiconductor materials include nitrides of Silicon, Gallium, Aluminum and Boron, and Zinc Selenide etc. in pure form or doped with elements such as Aluminum and Indium etc. Additionally, phosphor coatings may also be deployed with the plurality of LEDs 110, to achieve a myriad of wavelengths, through interference of primary wavelengths. An example for the material of phosphor coating includes Cerium doped Yttrium Aluminum Garnet.

However, in several embodiments, the plurality of LEDs 110 have been provided on a flexible Organic Light Emitting Diode (OLED) based films. The flexible OLED films use organic material based films to generate radiations in varying wavelengths. The flexibility of the OLED films would allow the utility garment 100 to be comparatively more ergonomic in usage by removing the rigidity induced by rigid LEDs. Alternately, the plurality of LEDs 110 may also be provided through flexible inorganic light emitting diode light sheets, strips, ribbons and tapes that may be installed within the utility garment 100 with application of pressure sensitive adhesives or other methods. Such ribbons and tapes may be made up of conductive materials such as copper and silver etc. An exemplary description of flexible inorganic light emitting diode strips can be found in granted United States Patent Numbered U.S. Pat. No. 7,476,557B2, titled “Roll-to-roll fabricated light sheet and encapsulated semiconductor circuit devices”, which is included herein in its entirety, by reference.

Physiological benefits of LED irradiation can be further studied from Opel D R, Hagstrom E, Pace A K, Sisto K, Hirano-Ali S A, Desai S, Swan J. Light-emitting Diodes: A Brief Review and Clinical Experience. J Clin Aesthet Dermatol. 2015 June; 8(6):36-44. PMID: 26155326; PMCID: PMC4479368, which is included herein in its entirety, by reference. Some of the key observations derived from the aforementioned art include:
1. Red LEDs specifically have been shown to activate fibroblast growth factor, increase type 1 pro-collagen, increase matrix metallo-proteinase-9 (MMP-9), and decrease MMP-1, thereby acting as an anti-aging agent.
2. Photomodulated yellow light alters ATP production, gene expression, and fibroblast activity. Increased ATP production is thought to be mediated via the absorption of photons by mitochondrial protoporphyrin IX. Interestingly, only photomodulated yellow LED has been shown to produce a tissue response implying that the light's ability to affect cells is dependent on the number and pattern of photon delivery.
3. Blue light appears to exert its effect on acne via its influence on Propionibacterium acnes and its anti-inflammatory properties. P. acnes contains naturally occurring porphyrins, mainly coproporphyrin and protoporphyrin IX. Absorption of blue light by these molecules is believed to induce a natural photodynamic therapy (PDT) effect with the destruction of the bacteria via the formation of oxygen free radicals. Blue light's anti-inflammatory effect appears to be the result of a shift in cytokine production.
4. Near infrared light, also known as monochromatic infrared energy (MIRE), is believed to stimulate circulation by inducing the release of guanylate cyclase and nitrous oxide, which, in turn, promotes vasodilation and growth factor production as well as angiogenesis, leading to subsequent wound healing.

The utility garment 100 also includes a plurality of sensors 112 configured to determine a plurality of parameters pertaining to the body of the user. In that regard, the plurality of sensors 112 includes motion sensors, humidity sensors and temperature sensors for the determination of parameters such as location, velocity, acceleration, heartbeat and perspiration of the body of the user. For example, the location sensors may deploy satellite navigation using any one or more of Global Positioning System (GPS), Global Navigation Satellite System (GLONASS), BeiDou Navigation Satellite System (BDS), Galileo, Quasi-Zenith Satellite System (QZSS) and Indian Regional Navigation Satellite System (IRNSS), also known as Navigation with Indian Constellation (NAVIC). Similarly, motion, velocity and direction of motion may be determined using combinations of accelerometers, gyroscopes and magnetometers. Additionally, the heartbeat can be determined using heart rate sensors deploying electrical means (generating an electrical signal on radio-detection of a heartbeat) or optical means (measuring scattering of visible light due to change of blood flow in blood vessels). Perspiration measurement for the body of the user can be obtained through sweat rate sensors that typically include a humidity chamber for collecting sweat and humidity sensors (for example, capacitive thin filmed humidity sensors), for determining the sweat rate. Also, the temperature sensors can be any one or more of, but not limited to, thermocouple or semiconductor based temperature sensors.

Further, an infrared heater 150 has been provided between the first layer 106 and the second layer 108 of the fabric 104. It is envisaged here that the infrared heater 150 be a far infrared type of heater. Typically, infrared heaters operate by providing long, medium and short-wave infrared radiations having wavelengths between 15 micrometers to 1 millimeter. Human skin absorbs far infrared radiations specifically well due to the skin composition comprising at least seventy percent of water. Additionally, the infrared heaters have an advantage of not giving off smell from dust, dirt, formaldehyde and toxic fumes from paint coating, etc. Therefore, they are suitable for human use as they are less likely to cause skin irritations and sensitivities.

The infrared heater 150 includes a plurality of flexible heating elements 152. The plurality of flexible heating elements 152 may be manufactured in the form of cords bundling several individual flexible strings. Such flexible strings may be made from carbon, or a ceramic material or a combination of ceramic material and carbon. The ceramic materials used in such applications typically utilize Mixed Metal Oxides (MMOs) that are compounds including oxides of two or more metals. Some of the exemplary metals used in MMOs include copper, cobalt, iron, trivalent chrome, tin, antimony, titanium, manganese and aluminum etc. Use of the ceramic materials at least in part ensures achieving higher emissivity as compared to using pure carbon alone as a heating material. It is also to be noted that heating of the plurality of flexible heating elements 152 is achieved through applying a potential difference along the plurality of flexible heating elements 152. The electrical resistance of the plurality of flexible heating elements 152 causes heat to be dissipated from the plurality of flexible heating elements 152.

FIG. 1B illustrates an exemplary arrangement of the plurality of flexible heating elements 152 of the infrared heater 150, in accordance with an embodiment of the present invention. It can be observed through FIG. 1B, that the plurality of flexible heating elements 152 are arranged in such a manner that they align with superficial veins of a portion of the body of the user, on which the utility garment 100 has been worn. Superficial veins are typically located parallel to body surface, in fat layer, between the skin and fascia covering muscle tissues. Since the utility garment 100 is illustrated to be a pair of track pants, the plurality of flexible heating elements 152 are arranged along great and small saphenous veins and tributaries thereof, (denoted by number 160) of the user. This is to ensure that while heating, the heat is directly supplied to veins causing the veins to expand and therefore allowing a greater flow of blood and supply of oxygen to several organs inside the body. Also, it is medically known that veins carry colder blood when compared to arteries, hence it would be intuitive to apply heating directly to the veins in order to speed up the blood flow to the heart of the user.

It is envisaged here that small adjustments may be possible in location of the plurality of flexible heating elements 152 in order to accommodate for varying body shapes and sizes of several potential users. In that manner, the plurality of flexible heating elements 152 have been located in a plurality of respective grooves in the fabric and a predetermined amount of clearance may be provided in the plurality of grooves of the fabric 104 to allow for the adjustment of the plurality of flexible heating elements 152. The elasticity and stretchability of the fabric 104 would allow the plurality of flexible heating elements 152 to remain in their respective locations, once the plurality of flexible heating elements 152 have been adjusted to their respective positions, by the user. In several embodiments, the plurality of flexible heating elements 152 may be connected with a dedicated power source (such as a battery or a terminal of a power module encapsulating the battery) through detachable connectors. Such detachable connectors would allow for the amount of current being supplied to the plurality of flexible heating elements 152, be controlled and convenient replacement in a situation where any one of the plurality of flexible heating elements 152 is damaged or dysfunctional.

Some additional discussion regarding the constructional features of the utility garment 100 has been provided in the following discussion. For example, the infrared heater 150 and the plurality of LEDs 110 are provided between the first layer 106 and the second layer 108 in order to protect the body or the skin of the user to come in direct contact with the infrared heater 150 and the plurality of LEDs 110. Also, the plurality of sensors 112 and the plurality of LEDs 110 are connected through a plurality of conductors 114.

FIG. 1C illustrates connection assembly of an LED 110a of the plurality of LEDs 110 with the plurality of conductors 114, in accordance with an embodiment 170 of the present invention. As illustrated in FIG. 1C, wherein the LED 110a is directly connected with the plurality of conductors 114, without the use of a Printed Circuit Board (PCB). The plurality of conductors 114 are envisaged to be flexible in nature. For example, the plurality of conductors 114 may be flexible threads of an electrically conducting material such as, but not limited to, Silver or its alloys. The plurality of conductors 114 in the form of the flexible threads may further be woven into the fabric 104. Further, it is envisaged that connecting terminals 111 of the plurality of LEDs 110 be made up of an electrically conducting material such as a metallic material and be pliable so as to allow them to form a loop around the passing plurality of conductors 114. This kind of setup allows the utility garment 100 to be relatively more flexible, as compared to an arrangement where relatively rigid PCBs would have been used to install the plurality of LEDs 110.

FIG. 1D illustrates connection arrangements 182, 184 of the plurality of LEDs 110, in accordance with an embodiment 180 of the present invention. While the arrangement 182 illustrates series connections of the plurality of LEDs 110 (110a, 110b, 110c, 110d . . . 110n), the arrangement 184 illustrates parallel connections of the plurality of LEDs 110 (110a, 110b, 110c, 110d . . . 110n). Each arrangement has its advantages and disadvantages and may be chosen in accordance with factors such as cost, specific application, electrical power available and efficiency of heat dissipation, etc.

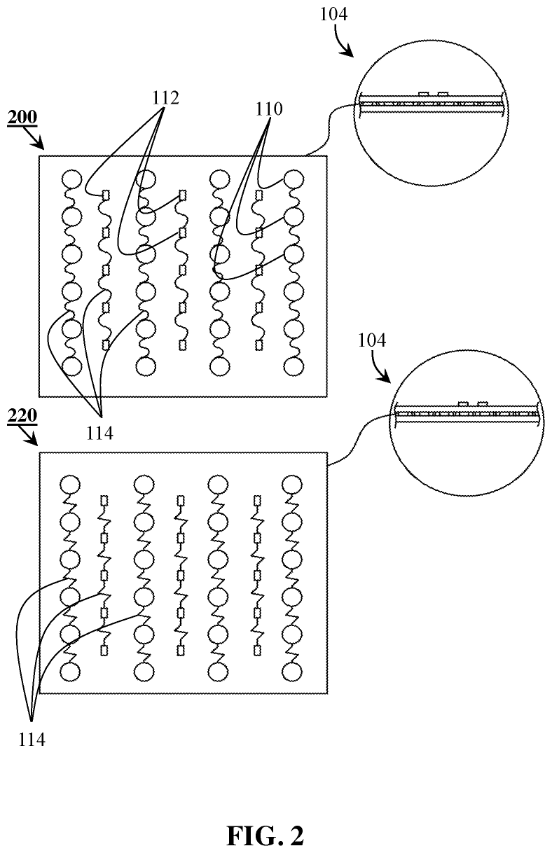

FIG. 2 illustrates two exemplary arrangements 200 and 220 of the plurality of conductors 114, within the utility garment 100, in accordance with an embodiment of the present invention. It is illustrated in FIG. 2, that the plurality of conductors 114 are arranged in predetermined patterns. For example, in the arrangement 200, the plurality of conductors 114 are arranged in ‘S’—shaped patterns. Similarly, in the arrangement 220, the plurality of conductors 114 are arranged in a zigzag pattern. The predetermined arrangements allow the plurality of conductors 114 to expand and contract, without breaking or straining, as the utility garment 100 is stretched and released during usage.

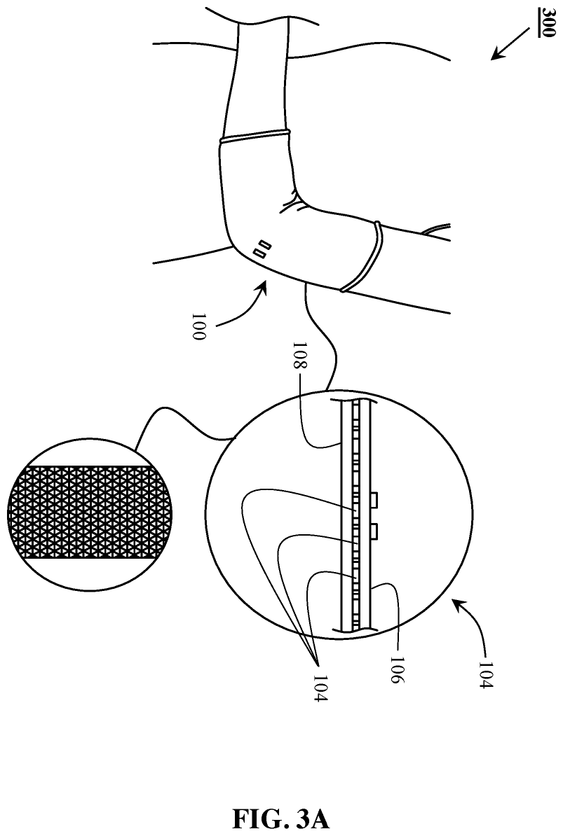

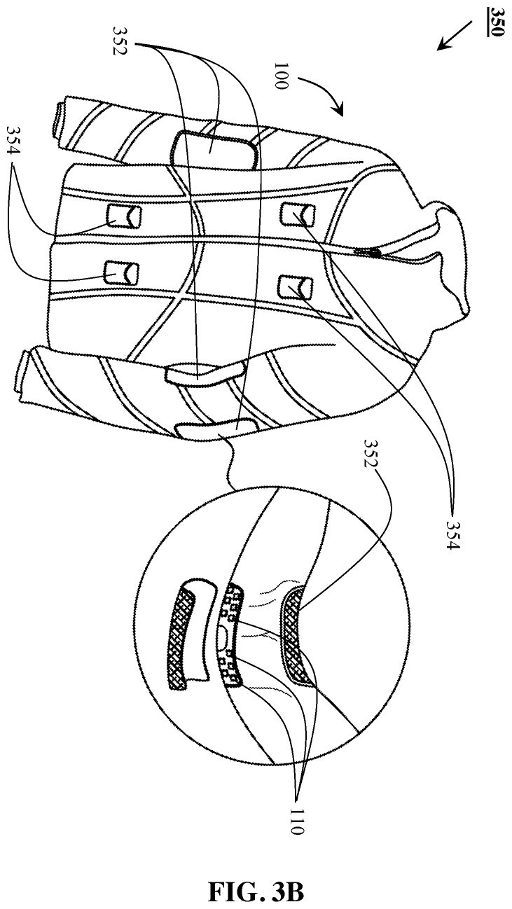

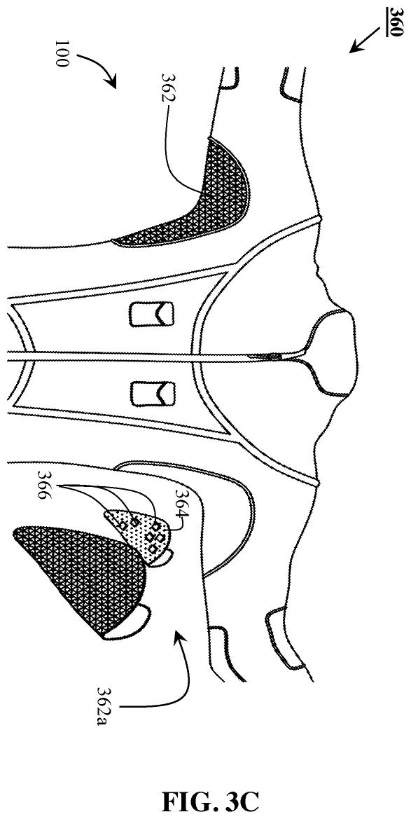

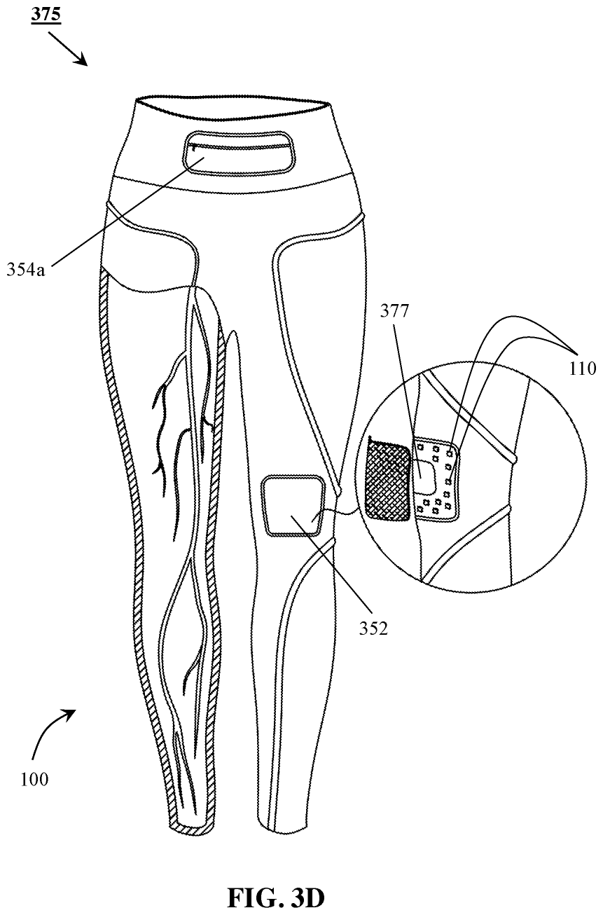

It has been previously emphasized that the utility garment 100 may not necessarily be the pair of track pants. In that regard, FIG. 3A illustrates the utility garment 100 in accordance with another embodiment 300 of the present invention. In this scenario, the utility garment 100 has been depicted as an arm gear. The features described above and in the following description are applicable to the same extent to the depicted arm gear as they apply to the pair of track pants. FIG. 3B illustrates the utility garment 100 in accordance with yet another embodiment 350 of the present invention. Here, the utility garment 100 has been depicted in the form of a jacket that may be worn on the upper portion of the body of the user. Similar to the arm gear, features described above and in the following description are applicable to the same extent to the depicted jacket as they apply to the pair of track pants.

It is further depicted in FIG. 3B, the utility garment 100 includes a plurality of auxiliary fabric portions 352. The plurality of auxiliary fabric portions 352 have been provided for covering joint areas in the body, in order to further enhance blood circulation in the joint areas (such as knees, shoulders, and elbows) by keeping the joint areas even warmer as compared to the muscle areas. In that regard, it is envisaged that the plurality of auxiliary fabric portions 352 be stretchable in nature and have comparatively higher heat insulating properties. One such fabric may be Spandex, also known as Lycra or Elastane in certain jurisdictions. However, the plurality of auxiliary fabric portions 352 may also be in form of zigzag or coiled fabric portions. Further, the plurality of auxiliary fabric portions 352 may include additional auxiliary infrared heating elements to increase localized heating. The plurality of auxiliary fabric portions 352 in that regard may be attached with the utility garment 100 through internal or external fusing and sewing or by sandwiching them between the first layer 106 and the second layer 108.

It is also illustrated in FIG. 3B, the utility garment 100 have been provided with a plurality of additional pockets 354. The plurality of additional pockets 354 have been provided to receive in them additional heating or cooling packages. Such packages may contain Phase Change Material (PCM) that create heating or cooling effect by release or absorption, respectively, of latent heat of fusion to or from the body. Some of commonly available PCMs include urea, ammonium nitrate, water, and other endothermic and exothermic materials. The heating or cooling packages may also be light (LED or laser etc.), resistive or Peltier effect based elements for active heating. In that regard, the plurality of additional pockets 354 may contain a power connector in order to supply power to received heating or cooling packages. In that regard, it is further envisaged that portions of the fabric 104 just under the plurality of additional pockets 354 have comparatively higher heat conduction properties. Further, closures for the plurality of additional pockets 354 may include zippers, loop and hook fasteners, magnets, buttons or snap fit closures etc.

FIG. 3C illustrates the utility garment 100 of FIG. 3B, in accordance with yet another embodiment 360 of the present invention. The utility garment 100 of FIG. 3C illustrates a plurality of moisture absorbing portions 362. The plurality of moisture absorbing portions 362 may be located at locations where the user is relatively more likely to release sweat from the body. As illustrated in FIG. 3C, the plurality of moisture absorbing portions 362 have been located at arm-pit areas of the utility garment 100. A moisture absorbing portion 362a at a right side of a front view of the utility garment 100 has been shown in an exploded view for clarity and elucidation. As illustrated, the moisture absorbing portion 362a includes a padding material 364 that may arrest the sweat of the user through absorption or adsorption depending upon construction and material properties of the padding material 364. In addition, the moisture absorbing portion 362 also includes a plurality of anti-microbial LEDs 366 that are envisaged to have both anti-bacterial and anti-fungal properties. The plurality of anti-microbial LEDs 366 are adapted to irradiate the body of the user for germicidal applications and prevent external and internal infections caused due to accumulation of sweat. The anti-microbial effects of Photodynamic Therapy (PDT) may be studied from Tegos George, Dai Tianhong, Fuchs Beth, Coleman Jeffrey, Prates Renato, Astrakas Christos, St Denis Tyler, Ribeiro Martha, Mylonakis Eleftherios, Hamblin Michael, “Concepts and Principles of Photodynamic Therapy as an Alternative Antifungal Discovery Platform”, Frontiers in Microbiology, Volume 3, 2012. Pg. 120, that is included herein, by reference, in its entirety. The provision of the plurality of moisture absorbing portions 362 allows the utility garment 100 to be used as sports and physical activity gear and would prevent the user from pathogenic conditions such as finger nail and toe nail fungus, Athlete's foot, jock itch, ringworm, and barber's itch etc.

FIG. 3D illustrates the utility garment 100 of FIG. 1B, in accordance with yet another embodiment 375 of the present invention. It is envisaged that at least some of the LEDs of the plurality of LEDs 110 may also be provided within the plurality of auxiliary fabric portions 352. But it is further envisaged that during bending of a joint, such as the knee joint or the elbow joint, the plurality of LEDs 110 do not line up along the bent joint. Therefore, a vacant portion 377 has been provided in areas of each one of the plurality of auxiliary fabric portions 352 that are directly over the bent joints, in order to prevent the plurality of LEDs 110 from relocating over to bent joint lines. A pocket 354a, of the plurality of additional pockets 354, with a zipper closure is also depicted in FIG. 3C. It is further envisaged that the plurality of additional pockets 354 be provided over high density tissue areas. The high density tissue areas are characterized by comparatively superior blood vessel distribution, heat conductivity and heat transport capability when compared with other areas in the body. Such high density tissue areas may include, for example, forearms, wrists, rib cage and upper torso etc. However, the plurality of additional pockets 354 may be provided at multiple locations in the utility garment 100 and their locations may be varied on a number of factors such as user demands, kind of applications and aesthetic concerns.

It is further envisaged that the utility garment 100 be capable of being controlled remotely, and be configured as per specifications desired by the user. Further, the utility garment 100 in itself should be able to carry out some preconfigured functions depending upon a specific application. In that manner, the utility garment 100 has been provided with a control architecture which will be discussed below. The control architecture has been elucidated only in a logical capacity, the actual construction and configurations may vary from one garment to other, based on factors such as, but not limited to, variations in height, weight and Body Mass Index (BMI) of the user, routine activities and sleeping patterns of the user, geographical locations and their corresponding climactic factors, where the utility garment 100 is being used, kind of applications such as yoga, outdoor sports and physical training, etc., type of garment (track pants or headgear or a jacket) and specific hardware/software/firmware functionalities as desired in a particular market.

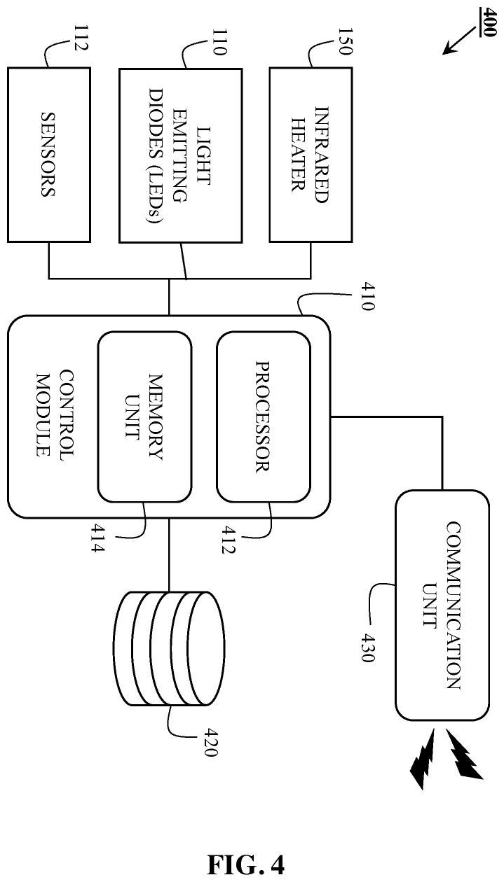

FIG. 4 illustrates a logical diagram of the control architecture provided with the utility garment 100, in accordance with an embodiment 400 of the present invention. The control architecture as depicted includes a control module 410 including a processor 412 and a memory unit 414. The processor 412 may be a general-purpose processor, a Field Programmable Gate Array (FPGA) or an Application Specific Integrated Circuit (ASIC), etc. Additionally, the memory unit 414 may be a volatile memory unit such as Static Random Access Memory (SRAM) and Dynamic Random Access Memory (DRAM) of types such as Asynchronous DRAM, Synchronous DRAM, Double Data Rate SDRAM, Rambus DRAM and Cache DRAM etc. The control module 410 is further connected with the infrared heater 150, the plurality of LEDs 110 and the plurality of sensors 112.

The control module 410 is further connected with a non-volatile storage device 420 which may be EPROM, EEPROM or flash memory based storage device. The control module 410 is also connected with a communication unit 430 that allows the utility garment 100 and more specifically the control module 410 to communicate with external devices. In that regard, the communication may through wired media such as those implementing IEEE 802.3 Ethernet standard or wireless media such as those implementing Bluetooth, Near Field Communication (NFC) and 802.11 Wireless Fidelity (Wi-Fi) or combinations thereof. In that regard, the communication unit 430 may include a port such as an Ethernet port or a Universal Serial Bus (USB) port or may be provided with a radio frequency transceiver.

The memory unit 414 may include machine readable instructions, that when executed by the processor 412, enables the processor 412 to determine magnitudes of the plurality of parameters including location, velocity, acceleration, heartbeat, and perspiration, through connection with the plurality of sensors 112. In several embodiments, the humidity sensors and the temperature sensors may be configured for measurements on or more of the body and ambient conditions. In that regard, it may be desired that both body measurements and ambient conditions (such as temperature and humidity in the immediate environment of the user) be factored while operating the utility garment 100. Also, the processor 412 is enabled to regulate the infrared heater 150 and the plurality of LEDs 110 in correlation with the determined magnitudes. For example, the processor 412 may be able to determine patterns of the motion, the heartbeat and the sweat rate of the user before a workout session, during the workout session and after the workout session and activate the infrared heater 150 and the plurality of LEDs 110 accordingly. For example, the user may need increased blood flow during a heavy workout session or the temperature of the skin of the user needs to be brought into a comfortable range of 1.5 to 3° C. of comfortable skin temperature of 33.4° C., before the work out session. Additionally, thermostats and thermistors may also be deployed in order to keep the skin temperature within a predetermined range.

In several embodiments, the processor 412 may further be enabled to regulate several treatment parameters such as intensity and dosage of heat and LED radiation, wavelengths of the LED radiation, mode of operation (pulsing or continuous) of the plurality of LEDs 110, time duration and combinations thereof. Generally accepted optimal clinical intensity for LED irradiation is 50-100 mW/cm². While it is envisaged that a large part of the control logic may already be programmed into the utility garment 100 in form of the machine readable instructions, it is further desired that at least some of the treatment parameters be controlled through an external device. Electrical power required to power the control architecture including the infrared heater 150, the plurality of LEDs 110 and the plurality of sensors 112 may be derived from onboard power sources such as rechargeable or replaceable batteries. The rechargeable batteries may be based on compositions such as Lithium-ion, Lithium-polymer, Nickel-metal-hydride and any other technology that may be made available in foreseeable future. However, the utility garment 100 may also be provided with Thermo-Electric Generator (TEG) based power sources.

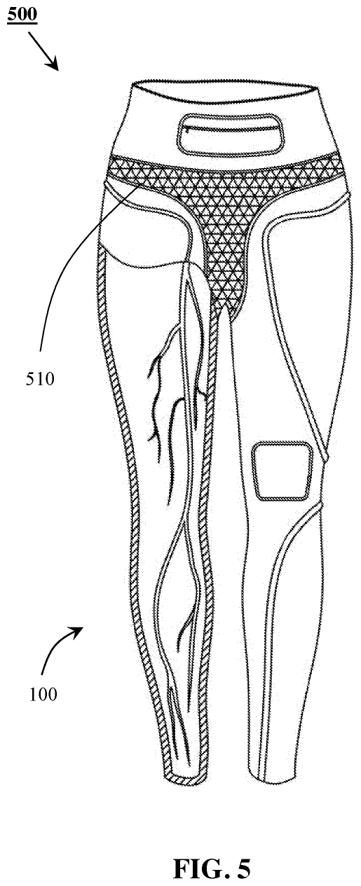

FIG. 5 illustrates a Thermo-Electric Generator (TEG) power source 510 for the utility garment 100, in accordance with an embodiment of the present invention. The objective of the TEG power source 510 is to harvest energy from the body and convert the harvested energy into electrical energy. To that end, the TEG power source 510 may be based on Eutectic Gallium Indium (EGaIn), where EGaIn liquid metal interconnects are encased in High Thermal Conductivity (HTC) elastomers. More information for the aforementioned construction can be obtained from the art Yasaman Sargolzaeiaval, Viswanath Padmanabhan Ramesh, Taylor V. Neumann, Veena Misra, Daryoosh Vashaee, Michael D. Dickey, Mehmet C. Öztürk, “Flexible thermoelectric generators for body heat harvesting—Enhanced device performance using high thermal conductivity elastomer encapsulation on liquid metal interconnects”, Applied Energy, Volume 262, 2020, 114370, ISSN 0306-2619, which is included herein by reference, in its entirety. EGaIn is a non-toxic alloy of gallium and indium that offers both stretchability and electrical conductivity. Further, the entire TEG power source 510 may be encapsulated into a silicone elastomer before attaching with the utility garment 100. However, the TEG power source 510 may also be made from several other techniques. For example, a TEG module may include ink-based thermo-elements made of nano-carbon bismuth telluride material, or HTC elastomers doped with EGaIn and graphene flakes.

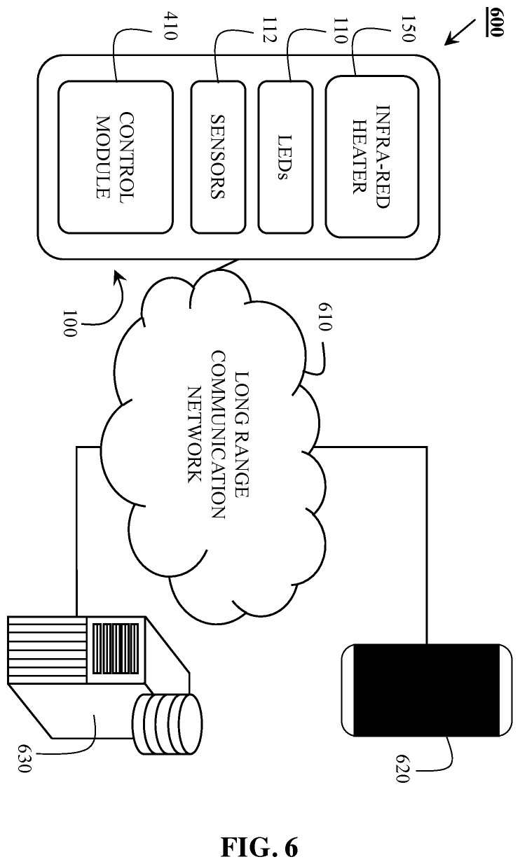

FIG. 6 illustrates a system 600 for a handheld device 620 based control of the utility garment 100, in accordance with an embodiment of the present invention. The handheld device 620 is envisaged to be mobile and may be a cellular phone, a smartphone, a tablet computer, a Personal Digital Assistant (PDA) or the like. In that manner, the utility garment 100 is able to communicate with the handheld device 620 using a long range communication network 610, either directly or through a remote server 630. The long range communication network 610 is envisaged to be a Wide Area Network (WAN) implemented through protocols such as those defined 802.x and 3GPP standards. For example, the long range communication network 610 may be Internet. The handheld device 620 may then able to communicate with the utility garment 100 through a thin client such as a web browser or a standalone mobile application.

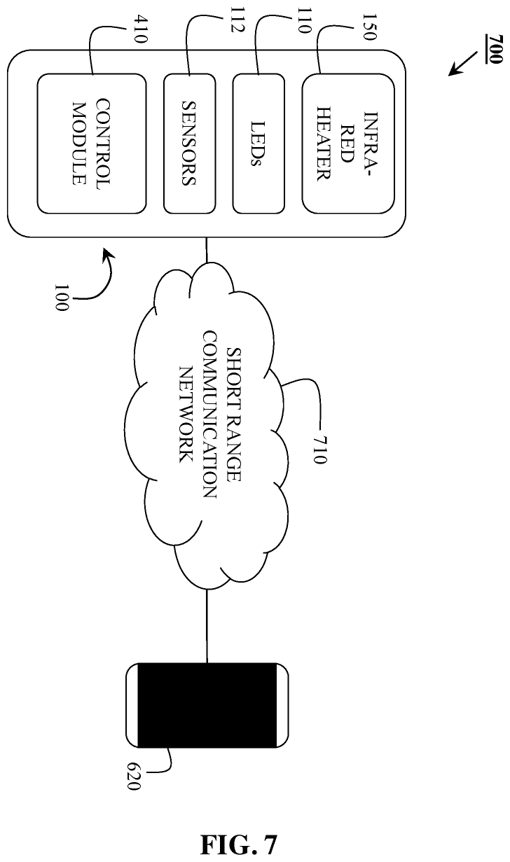

FIG. 7 illustrates a system 700 for the handheld device 620 based control of the utility garment 100, in accordance with another embodiment of the present invention. In this scenario, it is envisaged that the handheld device 620 may be able communicate with the utility garment 100 through a short range communication network 710, implemented through protocols such as Bluetooth, ZigBee, NFC or the like. In either of the two scenarios, the handheld device 620 may be able to provide a control signal to the control module 410 in order to alter any one or more of the treatment parameters on receiving such input from the user. For example, the user may be able to set the plurality of treatment parameters within low, medium and high ranges through the control signal transmitted to the control module 410, from the handheld device 620. Coarse and fine adjustments of the plurality of treatment parameters may also be possible via the handheld device 620.

During utilization, a user may wear the utility garment 100, for example in form of a pair of track pants for a physical training session. In that regard, the control module 410 may determine from usage history of the user that it is a time of the physical training for the user. Information available through the plurality of sensors 112 may also be utilized by the control module 410. Such information may include time of the day, location, motion and acceleration of the user, heart rate of the user, the skin temperature of the user, ambient conditions in immediate environment of the user and another factor such as BMI of the user. The control module 410 may in that regard activate one or more of the infrared heater 150, the plurality of LEDs 110 and the plurality of auxiliary fabric portion 352 in the joint areas, to increase the blood flow, relax muscle tissues and optimize skin temperature of the user, before the physical training session. The infrared heater 150 comprising plurality of flexible heating elements 152 may be adjusted over veins of the user due to the clearances provided in the plurality of grooves. Alternately, the user may also be able to regulate the treatment parameters through the handheld device 620, by connecting with the utility garment 100 over Internet or through Bluetooth or NFC etc. In that regard, the user may be able to connect to the utility garment 100 through a standalone application or a web browser installed with the handheld device 620.

During, the physical training session, increased blood flow may be required, while skin temperature may need to be regulated to not exceed comfortable limits. In that regard, the control module 410 may regulate the treatment parameters such as intensity and dosage of heat and LED radiation, wavelengths of the LED radiation, mode of operation (pulsing or continuous), time duration and combinations thereof. Moreover, the plurality of moisture absorbing portions 362 may arrest sweat produced during the physical training session in the padding material 364, through absorption or adsorption, and the plurality of anti-microbial LEDs 366 may be activated to prevent growth of fungal or bacterial infections. After the physical training session, the infrared heater 150 may be monitored by the control module 410 or by the user through the handheld device 620 to gradually bring the skin temperature within the comfortable range. Depending upon the ambient conditions and other aforementioned factors, the plurality of additional pockets 354 may be provided with additional heating or cooling packages, before, during or after the physical training session, to again maintain the body temperature of the user within a comfortable range.

The present invention as described above offers a number of advantages. First, the utility garment may be woven in multiple forms as required by several applications, due to flexibility in connections. The alignment of the flexible heating elements of the infrared heater with the superficial veins of the user allows heat to be transferred directly to the veins of the user, thus augmenting the blood circulation. Further, several treatment parameters such as intensity of heat and irradiation, operational modes and durations may either be programmed into a control logic provided within the utility garment or may be altered by the user using an external device, such as a handheld device.

The programming instructions can be, for example, computer executable and/or logic implemented instructions. In some examples, a computing device is configured to provide various operations, functions, or actions in response to the programming instructions conveyed to the computing device by one or more of the computer readable medium, the computer recordable medium, and/or the communications medium. The non-transitory computer readable medium can also be distributed among multiple data storage elements, which could be remotely located from each other. The computing device that executes some or all of the stored instructions can be a micro-fabrication controller, or another computing platform. Alternatively, the computing device that executes some or all of the stored instructions could be remotely located computer system, such as a server.

Further, while one or more operations have been described as being performed by or otherwise related to certain modules, devices or entities, the operations may be performed by or otherwise related to any module, device or entity. As such, any function or operation that has been described as being performed by a module could alternatively be performed by a different server, by the cloud computing platform, or a combination thereof.

Further, the operations need not be performed in the disclosed order, although in some examples, an order may be preferred. Also, not all functions need to be performed to achieve the desired advantages of the disclosed system and method, and therefore not all functions are required.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the invention is anticipated to hold on to all other such alternatives, modifications, and variations that fall within the scope of the present invention and appended claims.

1. A utility garment, comprising:
a first layer and a second layer of a fabric;
an infrared heater including a plurality of flexible heating elements, provided between the first layer and the second layer of the fabric;
a plurality of Light Emitting Diodes (LEDs) provided between the first and the second layer of the fabric; and
a plurality of sensors configured to determine a plurality of parameters pertaining to a body by which the utility garment has been worn.

2. The utility garment as claimed in claim 1, wherein the plurality of sensors and the plurality of LEDs are connected through a plurality of conductors arranged in one or more predetermined patterns.

3. The utility garment as claimed in claim 2, wherein the plurality of conductors are woven in the fabric.

4. The utility garment as claimed in claim 2, wherein the plurality of conductors are flexible threads of an electrically conducting material and connecting terminals of the plurality of LEDs are pliable so as to allow the connecting terminals to form a loop around the passing plurality of conductors.

5. The utility garment as claimed in claim 1, wherein the plurality of LEDs are provided on one or more of flexible Organic LED (OLED) and inorganic LED based films.

6. The utility garment as claimed in claim 1, wherein the plurality of flexible heating elements are located in a plurality of respective grooves in the fabric and a predetermined amount of clearance has been provided in the plurality of grooves of the fabric, to allow for the adjustment of the plurality of flexible heating elements.

7. The utility garment as claimed in claim 6, wherein the plurality of LEDs are connected in one or more of series connections, parallel connections and combinations thereof.

8. The utility garment as claimed in claim 1, further comprising a plurality of auxiliary fabric portions for covering joint areas in the body.

9. The utility garment as claimed in claim 8, wherein the plurality of auxiliary fabric portions include a plurality of auxiliary infrared heating elements.

10. The utility garment as claimed in claim 8, wherein the plurality of auxiliary fabric portions are stretchable in nature and are provided in one or more of straight, zigzag and coiled forms or combinations thereof.

11. The utility garment as claimed in claim 8, wherein each one of the plurality of auxiliary fabric portions includes a respective vacant portion, in order to prevent included plurality of LEDs from relocating over to joint lines.

12. The utility garment as claimed in claim 1, wherein the plurality of flexible heating elements is arranged in order to align with a plurality of veins in the body.

13. The utility garment as claimed in claim 1, further comprising a plurality of additional pockets in order to receive one or more of additional heating and cooling packages.

14. The utility garment as claimed in claim 13, wherein the one or more of additional heating and cooling packages include Phase Change Materials (PCMs).

15. The utility garment as claimed in claim 13, wherein the plurality of additional pockets include power connectors in order to supply power to the received one or more of additional heating and cooling packages.

16. The utility garment as claimed in claim 1, further comprising a plurality of moisture absorbing portions, each one of the plurality of moisture absorbing portions including:
a padding material adapted to arrest sweat through one or more of absorption and adsorption; and
a plurality of anti-microbial LEDs adapted to irradiate the body for germicidal applications.

17. The utility garment as claimed in claim 1, wherein the infrared heater, the plurality of LEDs and the plurality of sensors are connected with a processor and a memory unit, the memory unit including machine readable instructions that when executed by the processor, enables the processor to:
determine magnitudes of the plurality of parameters through connection with the plurality of sensors; and
regulate the infrared heater and the plurality of LEDs in correlation with the determined magnitudes.

18. The utility garment as claimed in claim 17, wherein the processor is further enabled to regulate a plurality of treatment parameters through the regulation of the infrared heater and the plurality of LEDs.

19. The utility garment as claimed in claim 17, wherein the processor is further enabled to communicate with a handheld device through one or more a long range communication network and a short range communication network.

20. The utility garment as claimed in claim 19, wherein the processor is further enabled to receive a control signal from the handheld device in order to set a plurality of treatment parameters within low, medium and high ranges.

21. The utility garment as claimed in claim 17, further comprising a power source configured to provide electrical power to the infrared heater, the plurality of LEDs and the plurality of sensors.

22. The utility garment as claimed in claim 21, wherein the power source includes a Thermo-Electric Generator (TEG) based power source.

23. The utility garment as claimed in claim 22, wherein TEG based power source includes Eutectic Gallium Indium (EGaIn) liquid metal interconnects encased in High Thermal Conductivity (HTC) elastomers.

24. The utility garment as claimed in claim 23, wherein the HTC elastomers are doped with graphene.

25. A method of utilizing a utility garment, the utility garment including an infrared heater, a plurality of LEDs and a plurality of sensors, the method comprising steps of:
determining magnitudes of a plurality of parameters pertaining to a body by which the utility garment has been worn, through connection with the plurality of sensors; and
regulating the infrared heater and the plurality of LEDs in correlation with the determined magnitudes.
